(19) United States
(12) Patent Application Publication
(10) Pub. No.: US 2024/0174628 A1
(43) Pub. Date: May 30, 2024

(54) METHOD FOR CONTINUOUSLY PREPARING CRUDE ETHYLENE SULFATE

(71) Applicant: Wuhan Oxiran Special Chemicals Company, Wuhan (CN)

(72) Inventors: Fan REN, Wuhan (CN); Liang WANG, Wuhan (CN); Wenchao SONG, Wuhan (CN); Rongming DAI, Wuhan (CN); Lichun LIANG, Wuhan (CN); Xianmiao CUI, Wuhan (CN)

(73) Assignee: Wuhan Oxiran Special Chemicals Company, Wuhan (CN)

(21) Appl. No.: 18/353,902

(22) Filed: Jul. 18, 2023

(30) Foreign Application Priority Data
Nov. 23, 2022 (CN) 202211477139.6

(51) Int. Cl. C07D 327/10; B01F 33/30; B01J 19/24

(52) U.S. Cl. CPC C07D 327/10; B01F 33/30; B01J 19/245; B01F 2101/2204; B01J 2219/0004

(57) ABSTRACT

In a method for continuously preparing crude ethylene sulfate, a sulfur trioxide solution is prepared by dissolving sulfur trioxide with a solution A, an ethylene oxide solution is prepared by mixing a solution B with ethylene oxide, the sulfur trioxide solution and the ethylene oxide solution are pre-cooled, and introduced into a set of microchannel reactors for a real-time reaction to obtain a mixed solution containing crude ethylene sulfate, and then a post-treatment process is carried out to obtain crude ethylene sulfate. With the process, the reaction selectivity is good, and a microchannel reaction can accurately control the reaction energy level due to its rapid mixing and timely heat transfer, which greatly reduces the safety risk and effectively avoids the occurrence of side reactions. One-step synthesis is realized, the atomic economic benefits are significantly improved, and thus the process is a typical low-carbon green chemical reaction.

### CROSS-REFERENCE TO RELATED APPLICATIONS

This application claims priority to Chinese Patent Application No. 202211477139.6 with a filing date of Nov. 23, 2022. The content of the aforementioned application, including any intervening amendments thereto, is incorporated herein by reference.

### TECHNICAL FIELD

The present disclosure relates to the technical field of compound synthesis, in particular to a method for continuously preparing crude ethylene sulfate.

### BACKGROUND

At present, there are many synthetic methods of ethylene sulfate (DTD), including acylation, substitution, addition, dioxane synthesis, oxidation, etc. Various reaction processes have advantages and disadvantages. For example, the acylation process has the advantage that a starting material is cheap and readily available, and the disadvantage that the reaction yield is low, and the starting material sulfuryl chloride or sulfuryl fluoride is a hazardous chemical and is highly corrosive.

The oxidation process is a primary synthesis method for electrolyte additive enterprises at present, a starting material is ethylene glycol, and reacts with dichlorosulfoxide to produce an intermediate vinyl sulfite, and vinyl sulfite is oxidized to form DTD. There are five main schemes for the oxidation process, wherein it is a commonly used method to obtain ethylene sulfate under the catalysis of a ruthenium trichloride aqueous solution by using sodium hypochlorite as an oxidant (scheme 1) at present.

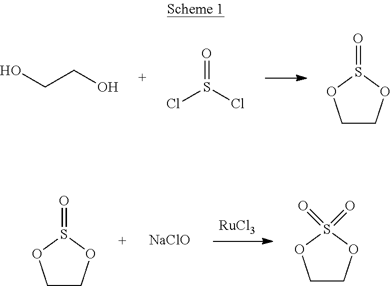

Due to the shorter development time of DTD, the process is not yet mature. In terms of cost, a noble metal catalyst ruthenium trichloride used in Scheme 1 is expensive and difficult to recycle; in terms of product indexes, sodium and chloride ion indexes in a product are likely to exceed the standard, affecting the application effect of the product; and in terms of waste, the use of excessive sodium hypochlorite as a strong oxidant produces a large amount of three wastes, resulting in a large amount of saline wastewater, which has a large impact on the environment.

In 1962, DOW published a patent: U.S. Pat. No. 3,045,027A proposed synthesis of DTD by a reaction of sulfur trioxide with ethylene oxide. However, due to the extremely active properties of reaction raw materials, this process has great safety risks in a kettle reaction. Therefore, there is an urgent need to develop a preparation process that is safe and reliable, has high conversion and high purity, and is suitable for large scale industrial production.

### SUMMARY OF PRESENT INVENTION

An objective of the present disclosure is to provide a method for continuously preparing crude ethylene sulfate with good treatment effects. DTD can be prepared efficiently and continuously using the method.

In one aspect, the present disclosure provides a method for continuously preparing crude ethylene sulfate, including the following steps of: preparing a sulfur trioxide solution by dissolving sulfur trioxide with a solution A, preparing an ethylene oxide solution by mixing a solution B with ethylene oxide, pre-cooling the sulfur trioxide solution and the ethylene oxide solution, introducing the pre-cooled sulfur trioxide solution and the pre-cooled ethylene oxide solution into a set of microchannel reactors for a real-time reaction to obtain a mixed solution containing crude ethylene sulfate, and then carrying out a post-treatment process to obtain crude ethylene sulfate.

A reaction equation for the method of the present disclosure is as follows:

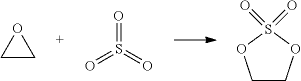

As a preference for the above technical solution, the method further includes some or all of the following technical features:

As an improvement of the above technical solution, a mass ratio of sulfur trioxide to ethylene oxide is 1:(0.5-1.5); the solution A is one or a mixture of more selected from a group consisting of dichloromethane, dichloroethane, trichloromethane, and carbon tetrachloride, and a solvent in the sulfur trioxide solution is 10%˜60% of a mass fraction of the solution; the solution B is one or a mixture of two selected from a group consisting of trichloromethane and carbon tetrachloride; and a solvent in the ethylene oxide solution is 0˜80% of a mass fraction of the solution.

As an improvement of the above technical solution, a reaction time in the set of the microchannel reactors is 5˜20 s, and a reaction pressure is 200˜1000 KPa.

As an improvement of the above technical solution, the sulfur trioxide solution and the ethylene oxide solution are cooled to −20° ° C. to 20° ° C. after a pre-cooling process.

As an improvement of the above technical solution, a mixer is used for a mixing process during preparing the sulfur trioxide solution by dissolving sulfur trioxide with the solution A, and preparing the ethylene oxide solution by mixing the solution B with ethylene oxide.

As an improvement of the above technical solution, the mixer is selected from a tubular in-line mixer or a microchannel in-line mixer, wherein the in-line mixer is preferably a static mixer, and the pre-cooling temperature after mixing is controlled to be −20° ° C. to 40° C.

As an improvement of the above technical solution, a reaction module of the microchannel reactor is made of silicon carbide, glass, stainless steel or ceramic, and a diaphragm pump is used for continuous feeding.

As an improvement of the above technical solution, the post-treatment process includes centrifugation, distillation, extraction, crystallization and filtration.

As an improvement of the above technical solution, the post-treatment process includes centrifugation, distillation, extraction, crystallization and filtration; wherein a solvent used for the extraction is one or more of ethanol, methanol, water, dioxane, dichloromethane, dichloroethane, diethyl ether, dimethyl carbonate, diethyl carbonate, and dioxolane, or a plurality of solvents are used for combined extraction.

In another aspect, the present disclosure further provides a device for continuously preparing crude ethylene sulfate which comprises a set of microchannel reactors comprising at least n microchannel reactors connected in series, wherein n=3-15; and two sets of mixers connected to the set of the microchannel reactors in parallel. The device for continuously preparing crude ethylene sulfate further includes a sulfur trioxide feeding system and an ethylene oxide feeding system. The sulfur trioxide feeding system employs a stainless steel constant temperature intermediate tank with a temperature of 30˜40° ° C., and a sealing material using a lining polytetrafluoroethylene or Hastelloy. The ethylene oxide feeding system employs a high-pressure low-temperature cooling intermediate tank with a pressure of 0.1˜2.0 Mpa and a temperature of 0˜30° ° C.

Compared with the prior art, the technical solution of the present disclosure has the following beneficial effects:

(1) The reaction selectivity is good, the microchannel conversion is more than 90%, and the gas chromatographic purity is more than 99.5%.

(2) A microchannel reaction can accurately control the reaction energy level due to its rapid mixing and timely heat transfer, which greatly reduces the safety risk and effectively avoids the occurrence of side reactions.

(3) The reaction steps are simplified to achieve one-step synthesis, the atomic economic benefits are significantly improved, and thus the process is a typical low-carbon green chemical reaction.

(4) The reaction time is significantly reduced from 5 hours in the traditional process to 5 s minimally, and the production efficiency is greatly improved. The above description is only the summary of the technical solution of the present disclosure, and can be implemented according to the contents of the specification in order to more clearly understand the technical means of the present disclosure, and in order to make the above and other purposes, features and advantages of the present disclosure more obvious and easy to understand, a detailed description is as follows in connection with the preferred embodiments.

### BRIEF DESCRIPTION OF THE DRAWINGS

In order to more clearly illustrate the technical solutions of the embodiments of the present disclosure, the drawings of the embodiments are briefly described below.

FIG. 1 is a diagram showing a process for continuously preparing crude ethylene sulfate according to the present disclosure;

FIG. 2 is an infrared spectrum of a product in Embodiment 1 of the present disclosure;

FIG. 3 is a gas chromatogram of the product in Embodiment 1 of the present disclosure;

FIG. 4 is a gas chromatogram of a product in Embodiment 2 of the present disclosure;

FIG. 5 is a gas chromatogram of a product in Embodiment 3 of the present disclosure;

FIG. 6 is a gas chromatogram of a product in Embodiment 4 of the present disclosure; and

FIG. 7 is a gas chromatogram of a product in Embodiment 5 of the present disclosure.

### DETAILED DESCRIPTION OF THE EMBODIMENTS

Specific implementations of the present disclosure are described in detail below, which, as a part of this specification, illustrate the principle of the present disclosure by the embodiments, and other aspects, features and advantages of the present disclosure will become apparent from this detailed description.

### Embodiment 1

150 Kg of a 30 wt % sulfur trioxide solution was prepared by dissolving sulfur trioxide with a solution A, 45 Kg of a 60% ethylene oxide solution was prepared by mixing a solution B with ethylene oxide, the sulfur trioxide solution and the ethylene oxide solution were pre-cooled to 10° C., the pre-cooled sulfur trioxide solution and the pre-cooled ethylene oxide solution were introduced into microchannel reactors while maintaining a mass flow rate of the sulfur trioxide solution at 15 Kg/min, and maintaining a mass flow rate of the ethylene oxide solution at 4.5 Kg/min for a reaction to obtain a mixed solution containing crude ethylene sulfate, wherein the reaction pressure was maintained to be 500 KPa or below, the reaction time was controlled to be 12 s, the number of reaction modules was 8, and the reaction temperature was controlled to be 10° C.

The obtained mixed solution of crude ethylene sulfate was injected into a continuous extraction device for continuous extraction with the temperature maintained at 20° C., namely, first water [the mixed solution of the crude product:water=1:1.5 (a mass ratio)] was added into a high-speed stirrer, high-speed stirring and uniform mixing were performed, centrifugation was conducted at a rotating speed controlled at 6000˜10000 revolutions for layering, and the above steps were repeatedly performed once, and an oil phase was retained.

Further, a mixed solution of methanol/water (a mass ratio of 1:1) (a ratio of the mixed solution to the oil phase being 1:2) was added into the high-speed stirrer, high-speed stirring and uniform mixing were performed, centrifugation was conducted at a rotating speed controlled at 6000˜10000 revolutions for layering, and an oil phase was retained.

The above steps were mainly used to remove by-products in the system: dioxane and linear sulfate type by-products. A solvent was removed from the oil phase, the remaining DTD crude product was 58.4 Kg, the calculated yield was about 80.7%, and the product purity was 99.583%.

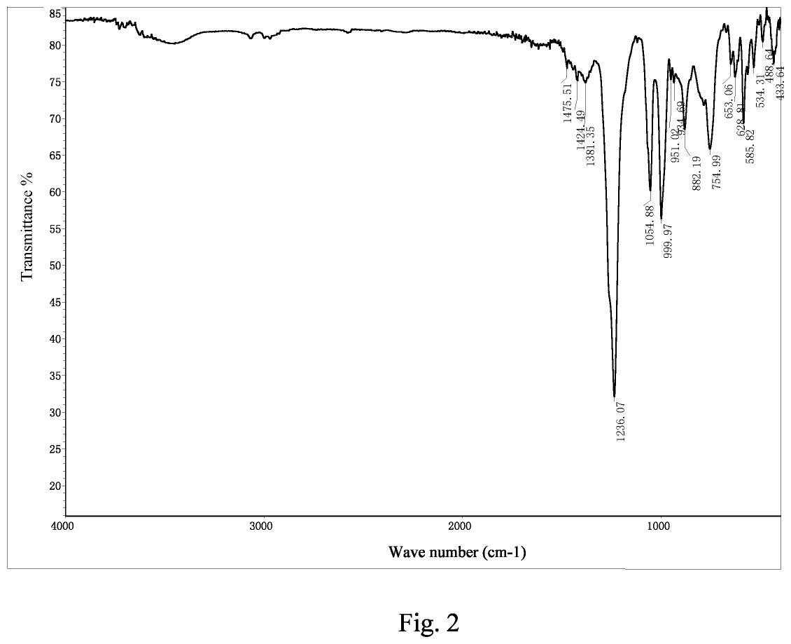

FIG. 2 is an infrared spectrum of a product in this embodiment. From the figure, it can be seen that its infrared spectrum shows characteristic absorption peaks at 1236 cm⁻¹, 1055 cm⁻¹, 1000 cm⁻¹, 882 cm⁻¹, 755 cm⁻¹ and 586 cm⁻¹.

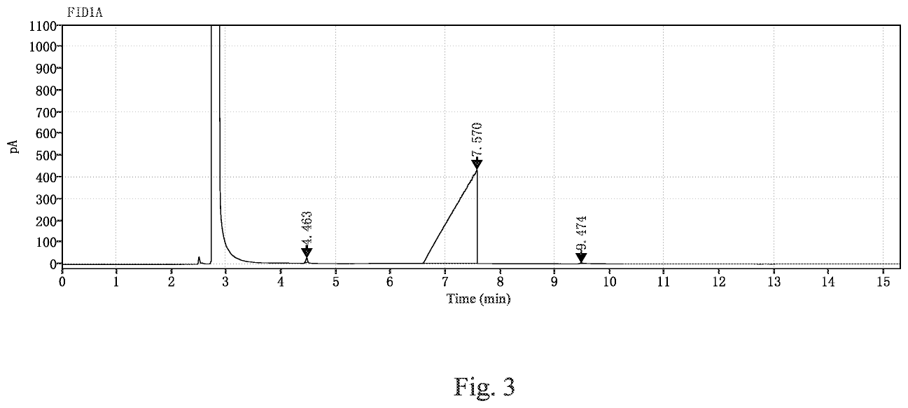

The product was subjected to gas chromatography, and the results are shown in Table 1, and FIG. 3 is a gas chromatogram of this gas chromatography.

TABLE 1
Gas chromatography data table of the product in Embodiment 1
Signal: FID1A

| Retention | | Peak width | | Peak | Peak |
|---|---|---|---|---|---|
| time (min) | Type | (min) | Peak area | height | area % |
| 4.463 | MM m | 0.08 | 42.92 | 20.77 | 0.333 |
| 7.570 | MB m | 1.01 | 12848.00 | 431.58 | 99.583 |
| 9.474 | MM m | 0.12 | 10.83 | 3.41 | 0.084 |
| | | Sum | 12901.74 | | |

### Embodiment 2

200 Kg of a 30 wt % sulfur trioxide solution was prepared by dissolving sulfur trioxide with a solution A, 60 Kg of a 60% ethylene oxide solution was prepared by mixing a solution B with ethylene oxide, the sulfur trioxide solution and the ethylene oxide solution were pre-cooled to 10° C., the pre-cooled sulfur trioxide solution and the pre-cooled ethylene oxide solution were introduced into microchannel reactors while maintaining a mass flow rate of the sulfur trioxide solution at 20 Kg/min, and maintaining a mass flow rate of the ethylene oxide solution at 6 Kg/min for a reaction to obtain a mixed solution containing crude ethylene sulfate, wherein the reaction pressure was maintained to be 500 KPa or below, the reaction time was controlled to be 12 s, the number of reaction modules was 8, and the reaction temperature was controlled to be 10° C.

The obtained mixed solution of crude ethylene sulfate was injected into a continuous extraction device for continuous extraction with the temperature maintained at 20° C., namely, first water [the mixed solution of the crude product:water=1:1.5 (a mass ratio)] was added into a high-speed stirrer, high-speed stirring and uniform mixing were performed, centrifugation was conducted at a rotating speed controlled at 6000˜10000 revolutions for layering, and the above steps were repeatedly performed once, and an oil phase was retained.

Further, a mixed solution of methanol/water (a mass ratio of 1:1) (a ratio of the mixed solution to the oil phase being 1:2) was added into the high-speed stirrer, high-speed stirring and uniform mixing were performed, centrifugation was conducted at a rotating speed controlled at 6000˜10000 revolutions for layering, and an oil phase was retained.

The above steps were mainly used to remove by-products in the system: dioxane and linear sulfate type by-products. A solvent was removed from the oil phase, the remaining DTD crude product was 79.6 Kg, the calculated yield was about 82.6%, and the product purity was 99.580%.

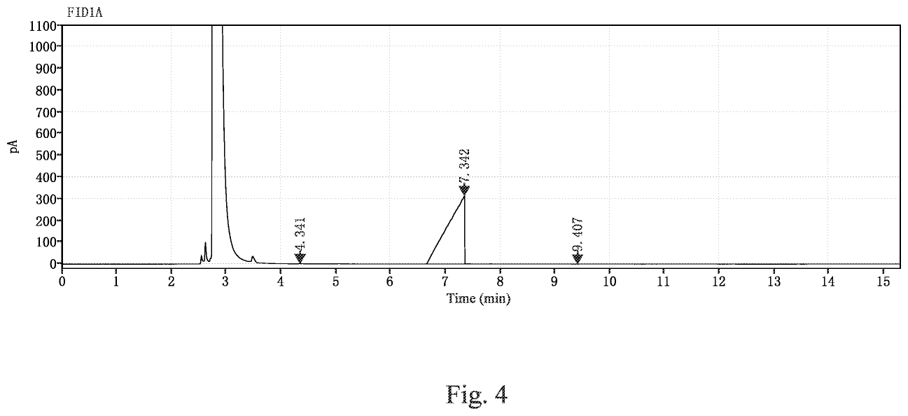

The product was subjected to gas chromatography, and the results are shown in Table 2, and FIG. 4 is a gas chromatogram of this gas chromatography.

TABLE 2
Gas chromatography data table of the product in Embodiment 2
Signal: FID1A

| Retention | | Peak width | | Peak | Peak |
|---|---|---|---|---|---|
| time (min) | Type | (min) | Peak area | height | area % |
| 4.341 | MV m | 0.30 | 17.62 | 2.16 | 0.263 |
| 7.342 | BV | 0.78 | 6666.95 | 314.68 | 99.580 |
| 9.407 | MM m | 0.21 | 10.47 | 0.77 | 0.156 |
| | | Sum | 6695.05 | | |

### Embodiment 3

300 Kg of a 30 wt % sulfur trioxide solution was prepared by dissolving sulfur trioxide with a solution A, 90 Kg of a 60% ethylene oxide solution was prepared by mixing a solution B with ethylene oxide, the sulfur trioxide solution and the ethylene oxide solution were pre-cooled to 10° C., the pre-cooled sulfur trioxide solution and the pre-cooled ethylene oxide solution were introduced into microchannel reactors while maintaining a mass flow rate of the sulfur trioxide solution at 30 Kg/min, and maintaining a mass flow rate of the ethylene oxide solution at 9 Kg/min for a reaction to obtain a mixed solution containing crude ethylene sulfate, wherein the reaction pressure was maintained to be 500 KPa or below, the reaction time was controlled to be 12 s, the number of reaction modules was 8, and the reaction temperature was controlled to be 10° C.

The obtained mixed solution of crude ethylene sulfate was injected into a continuous extraction device for continuous extraction with the temperature maintained at 20° ° C., namely, first water [the mixed solution of the crude product:water=1:1.5 (a mass ratio)] was added into a high-speed stirrer, high-speed stirring and uniform mixing were performed, centrifugation was conducted at a rotating speed controlled at 6000˜10000 revolutions for layering, and the above steps were repeatedly performed once, and an oil phase was retained.

Further, a mixed solution of methanol/water (a mass ratio of 1:1) (a ratio of the mixed solution to the oil phase being 1:2) was added into the high-speed stirrer, high-speed stirring and uniform mixing were performed, centrifugation was conducted at a rotating speed controlled at 6000˜10000 revolutions for layering, and an oil phase was retained.

The above steps were mainly used to remove by˜products in the system: dioxane and linear sulfate type by˜products. A solvent was removed from the oil phase, the remaining DTD crude product was 118.5 Kg, the calculated yield was about 81.9%, and the product purity was 99.493%.

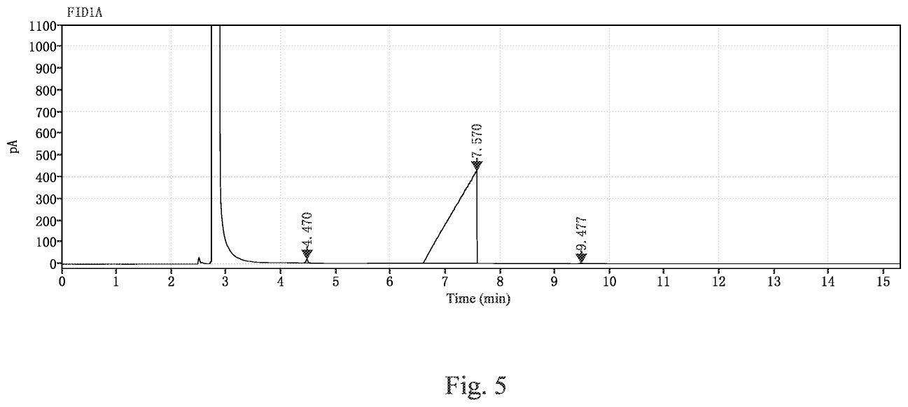

The product was subjected to gas chromatography, and the results are shown in Table 3, and FIG. 5 is a gas chromatogram of this gas chromatography.

TABLE 3
Gas chromatography data table of the product in Embodiment 3
Signal: FID1A

| Retention | | Peak width | | Peak | Peak |
|---|---|---|---|---|---|
| time (min) | Type | (min) | Peak area | height | area % |
| 4.470 | BV | 0.22 | 50.30 | 18.28 | 0.392 |
| 7.570 | MB m | 1.09 | 12768.38 | 426.15 | 99.493 |
| 9.477 | BV | 0.26 | 14.75 | 2.27 | 0.115 |
| | | Sum | 12833.44 | | |

### Embodiment 4

200 Kg of a 30 wt % sulfur trioxide solution was prepared by dissolving sulfur trioxide with a solution A, 60 Kg of a 60% ethylene oxide solution was prepared by mixing a solution B with ethylene oxide, the sulfur trioxide solution and the ethylene oxide solution were pre-cooled to 0° C., the pre-cooled sulfur trioxide solution and the pre-cooled ethylene oxide solution were introduced into microchannel reactors while maintaining a mass flow rate of the sulfur trioxide solution at 20 Kg/min, and maintaining a mass flow rate of the ethylene oxide solution at 6 Kg/min for a reaction to obtain a mixed solution containing crude ethylene sulfate, wherein the reaction pressure was maintained to be 500 KPa or below, the reaction time was controlled to be 12 s, the number of reaction modules was 8, and the reaction temperature was controlled to be 0° C.

The obtained mixed solution of crude ethylene sulfate was injected into a continuous extraction device for continuous extraction with the temperature maintained at 20° C., namely, first water [the mixed solution of the crude product:water=1:1.5 (a mass ratio)] was added into a high-speed stirrer, high-speed stirring and uniform mixing were performed, centrifugation was conducted at a rotating speed controlled at 6000˜10000 revolutions for layering, and the above steps were repeatedly performed once, and an oil phase was retained.

Further, a mixed solution of methanol/water (a mass ratio of 1:1) (a ratio of the mixed solution to the oil phase being 1:2) was added into the high-speed stirrer, high-speed stirring and uniform mixing were performed, centrifugation was conducted at a rotating speed controlled at 6000˜10000 revolutions for layering, and an oil phase was retained.

The above steps were mainly used to remove by-products in the system: dioxane and linear sulfate type by-products. A solvent was removed from the oil phase, the remaining DTD crude product was 79.7 Kg, the calculated yield was about 82.7%, and the product purity was 99.626%.

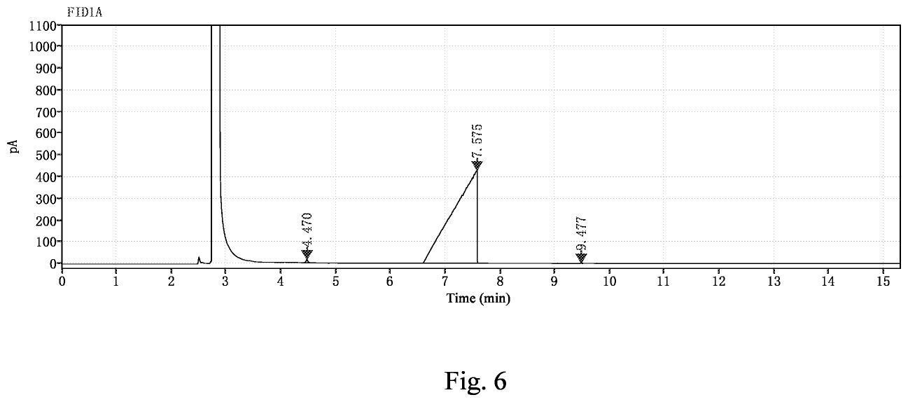

The product was subjected to gas chromatography, and the results are shown in Table 4, and FIG. 6 is a gas chromatogram of this gas chromatography.

TABLE 4
Gas chromatography data table of the product in Embodiment 4
Signal: FID1A

| Retention | | Peak width | | Peak | Peak |
|---|---|---|---|---|---|
| time (min) | Type | (min) | Peak area | height | area % |
| 4.470 | BB | 0.28 | 40.46 | 14.67 | 0.313 |
| 7.575 | BB m | 1.06 | 12860.27 | 425.70 | 99.626 |
| 9.477 | BV | 0.23 | 7.83 | 1.20 | 0.061 |
| | | Sum | 12908.57 | | |

### Embodiment 5

200 Kg of a 30 wt % sulfur trioxide solution was prepared by dissolving sulfur trioxide with a solution A, 60 Kg of a 60% ethylene oxide solution was prepared by mixing a solution B with ethylene oxide, the sulfur trioxide solution and the ethylene oxide solution were pre-cooled to 20° C., the pre-cooled sulfur trioxide solution and the pre-cooled ethylene oxide solution were introduced into microchannel reactors while maintaining a mass flow rate of the sulfur trioxide solution at 20 Kg/min, and maintaining a mass flow rate of the ethylene oxide solution at 6 Kg/min for a reaction to obtain a mixed solution containing crude ethylene sulfate, wherein the reaction pressure was maintained to be 500 KPa or below, the reaction time was controlled to be 12 s, the number of reaction modules was 8, and the reaction temperature was controlled to be 20° C.

The obtained mixed solution of crude ethylene sulfate was injected into a continuous extraction device for continuous extraction with the temperature maintained at 20° C., namely, first water [the mixed solution of the crude product:water=1:1.5 (a mass ratio)] was added into a high-speed stirrer, high-speed stirring and uniform mixing were performed, centrifugation was conducted at a rotating speed controlled at 6000˜10000 revolutions for layering, and the above steps were repeatedly performed once, and an oil phase was retained.

Further, a mixed solution of methanol/water (a mass ratio of 1:1) (a ratio of the mixed solution to the oil phase being 1:2) was added into the high-speed stirrer, high-speed stirring and uniform mixing were performed, centrifugation was conducted at a rotating speed controlled at 6000˜10000 revolutions for layering, and an oil phase was retained.

The above steps were mainly used to remove by-products in the system: dioxane and linear sulfate type by-products. A solvent was removed from the oil phase, the remaining DTD crude product was 80.4 Kg, the calculated yield was about 83.4%, and the product purity was 99.690%.

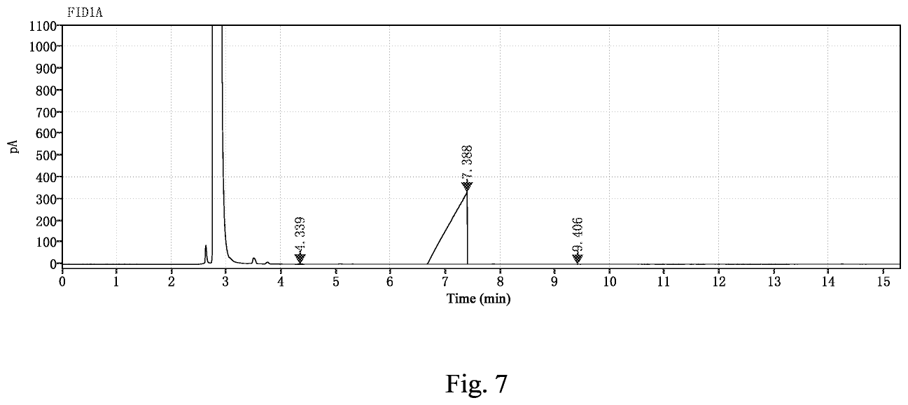

The product was subjected to gas chromatography, and the results are shown in Table 5, and FIG. 7 is a gas chromatogram of this gas chromatography.

TABLE 5
Gas chromatography data table of the product in Embodiment 5
Signal: FID1A

| Retention | | Peak width | | Peak | Peak |
|---|---|---|---|---|---|
| time (min) | Type | (min) | Peak area | height | area % |
| 4.339 | MM m | 0.16 | 17.13 | 2.90 | 0.232 |
| 7.388 | BV | 0.79 | 7361.40 | 332.84 | 99.690 |
| 9.406 | MM m | 0.09 | 5.79 | 0.87 | 0.078 |
| | | Sum | 7384.32 | | |

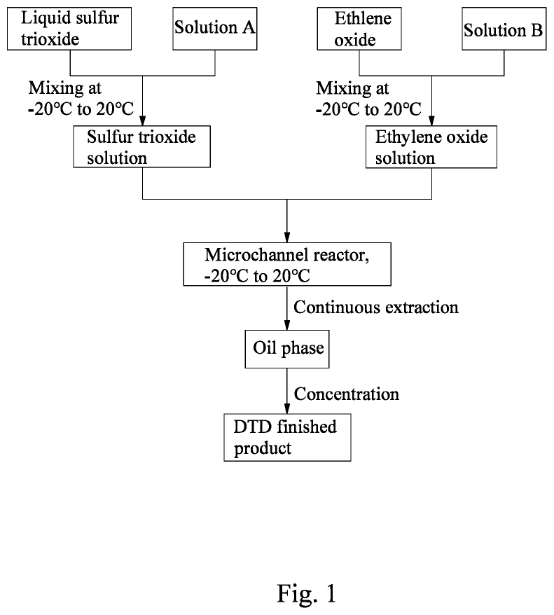

As shown in FIG. 1, which is a schematic diagram of the method for continuously preparing crude ethylene sulfate according to the preferred embodiments of the present disclosure, the method for continuously preparing crude ethylene sulfate of the present disclosure has good reaction selectivity, a conversion rate of more than 98%, and a yield of more than 95%, the purity of the crude product is more than 90%, the electronic grade purity is more than 99.8%, the moisture content is less than 30 ppm, and an acid number is less than 50 ppm. A microchannel reaction can accurately control the reaction energy level due to its rapid mixing and timely heat transfer, which greatly reduces the safety risk and effectively avoids the occurrence of side reactions. The reaction steps are simplified to achieve one-step synthesis, the atomic economic benefits are significantly improved, and thus the process is a typical low-carbon green chemical reaction. The reaction time is significantly reduced from 5 hours in the traditional process to 5 s minimally, and the production efficiency is greatly improved.

The raw materials listed in the present disclosure, the upper and lower limits and interval values of the raw materials in the present disclosure, and the upper and lower limits and interval values of process parameters (such as the temperature, time, etc.) can realize the present disclosure, and the embodiments are not listed here.

The above are only preferred embodiments of the present disclosure, and of course, cannot be intended to limit the scope of the present disclosure. It should be noted that for those of ordinary skill in the art, several improvements and changes can be made without departing from the principle of the present disclosure, and these improvements and changes are also considered to be within the scope of protection of the present disclosure.

What is claimed is:

1. A method for continuously preparing crude ethylene sulfate, comprising the following steps of: preparing a sulfur trioxide solution by dissolving sulfur trioxide with a solution A, preparing an ethylene oxide solution by mixing a solution B with ethylene oxide, pre-cooling the sulfur trioxide solution and the ethylene oxide solution, introducing the pre-cooled sulfur trioxide solution and the pre-cooled ethylene oxide solution into a set of microchannel reactors for a real-time reaction to obtain a mixed solution containing crude ethylene sulfate, and then carrying out a post-treatment process to obtain the crude ethylene sulfate.

2. The method according to claim 1, wherein a mass ratio of sulfur trioxide to ethylene oxide is 1:(0.5˜1.5); the solution A is one or a mixture of more selected from a group consisting of dichloromethane, dichloroethane, trichloromethane, and carbon tetrachloride, and a solvent in the sulfur trioxide solution is 10%˜60% of a mass fraction of the solution; the solution B is one or a mixture of two selected from a group consisting of trichloromethane and carbon tetrachloride; and a solvent in the ethylene oxide solution is 0˜80% of a mass fraction of the solution.

3. The method according to claim 1, wherein a reaction time in each microchannel reactor of the set of the microchannel reactors is 5˜20 s, and a reaction pressure is 200˜1000 Kpa.

4. The method according to claim 1, wherein the sulfur trioxide solution and the ethylene oxide solution are cooled to −20° C. to 20° ° C. after the pre-cooling process.

5. The method according to claim 1, wherein a mixer is used for a mixing process during preparing the sulfur trioxide solution by dissolving sulfur trioxide with the solution A, and preparing the ethylene oxide solution by mixing the solution B with ethylene oxide.

6. The method according to claim 5, wherein the mixer is selected from a tubular in-line mixer or a microchannel in-line mixer, the in-line mixer is a static mixer, and a pre-cooling temperature after mixing is controlled to be −20° ° C. to 40° C.

7. The method according to claim 1, wherein a reaction module of the microchannel reactors is made of silicon carbide, glass, stainless steel or ceramic, and a diaphragm pump is used for continuous feeding.

8. The method according to claim 1, wherein the post-treatment process comprises centrifugation, distillation, extraction, crystallization and filtration.

9. The method according to claim 1, wherein the post-treatment process comprises centrifugation, distillation, extraction, crystallization and filtration; a solvent used for the extraction is one or more selected from a group consisting of ethanol, methanol, water, dioxane, dichloromethane, dichloroethane, diethyl ether, dimethyl carbonate, diethyl carbonate, and dioxolane, or a plurality of solvents are used for combined extraction.

10. A device for continuously preparing crude ethylene sulfate, comprising:
a set of microchannel reactors comprising at least n microchannel reactors connected in series, wherein n=3-15; and
two sets of mixers are connected to the set of microchannel reactors in parallel.
